(19) United States
(12) Patent Application Publication
(10) Pub. No.: US 2017/0325691 A1
(43) Pub. Date: Nov. 16, 2017

(54) ACOUSTIC WAVE RECEIVING APPARATUS

(71) Applicant: CANON KABUSHIKI KAISHA, Tokyo (JP)

(72) Inventor: Hiroshi Nishihara, Kawasaki-shi (JP)

(21) Appl. No.: 15/584,115

(22) Filed: May 2, 2017

(30) Foreign Application Priority Data
May 10, 2016 (JP) 2016-094477
May 2, 2017 (JP) 2017-091603

(51) Int. Cl. A61B 5/00

(52) U.S. Cl. CPC A61B 5/0095

(57) ABSTRACT

Disclosed is an acoustic wave receiving apparatus including: a light irradiation unit; a receiving array receiving photoacoustic waves generated by a subject portion to output photoacoustic signals; a transmitting and receiving array transmitting ultrasound waves to the subject portion and receiving echo waves to output ultrasound signals; an array support unit having a first portion supporting the receiving array a second portion supporting the transmitting and receiving array; a scanning unit integrally scanning the arrays. The array support unit is so configured that effective reception region and a focusing region formed by the transmitting and receiving array do not overlap each other.

### BACKGROUND OF THE INVENTION

### Field of the Invention

The present invention relates to an acoustic wave receiving apparatus.

### Description of the Related Art

Research on an optical imaging apparatus that emits light from a light source such as a laser onto a subject portion such as a living body and converts information inside the subject portion obtained based on the incident light into an image has been actively promoted in the field of medicine. As one of optical imaging technologies, photoacoustic imaging (PAI) has been known. In the photoacoustic imaging, pulsed light generated from a light source is irradiated onto a subject portion, acoustic waves (typically ultrasound waves) generated from the tissues of the subject portion having absorbed the energy of the pulsed light propagating and spreading inside the subject portion are received, and information on the subject portion is converted into an image (imaging) based on the receiving signals of the acoustic waves.

When light is irradiated onto a subject portion, the subject portion having absorbed the energy of the light instantaneously expands and generates acoustic waves (called photoacoustic waves) due to a difference in the absorptivity of the energy of the light between a target region such as a tumor and the other tissues. In the photoacoustic imaging, photoacoustic waves generated by this photoacoustic effect are received using a probe (receiving elements).

By mathematically analyzing the receiving signals, it is possible to acquire information inside a subject portion, particularly an initial sound pressure distribution, a light energy absorption density distribution, an absorption coefficient distribution, or the like. These information items may also be used to quantitatively measure a specific substance inside a subject portion, e.g., oxygen saturation inside blood or the like. In recent years, using this photoacoustic imaging, preclinical studies for taking images of the vessels of small animals and clinical studies for applying this principle to diagnosis for breast cancer or the like have been actively promoted.

“Dedicated 3D Photoacoustic Breast Imaging” by Robert A Kruger, Cherie M. Kuzmiak, Richard B. Lam, Daniel R. Reinecke, Stephen P. Del Rio, and Doreen Steed, Medical Physics 40, 113301 (2013) describes an acoustic wave receiving apparatus that acquires subject-portion information using a sensor in which the receiving surfaces of a plurality of receiving elements are arranged on the inner surface of a hemispherical shell-shaped array support unit. Since this sensor is capable of receiving photoacoustic waves generated from a specific region with high sensitivity, the resolution of the information on the subject portion in the specific region is improved.

In addition, in the acoustic wave receiving apparatus described in the above “Dedicated 3D Photoacoustic Breast Imaging,” an acoustic matching fluid made of a liquid or a gel is filled inside the hemispherical shell-shaped array support unit to make acoustic waves propagate through an ultrasound probe. Further, the sensor is moved in a horizontal direction with respect to the subject portion immersed in the acoustic matching fluid to acquire the information on the subject portion at high resolution in a wide range.

Note that a container-shaped sensor in which an acoustic matching fluid is held is available not only for photoacoustic imaging but also for ultrasound echo diagnosis. When such a sensor is used, an acoustic matching fluid is preferably filled between a subject portion and the sensor to acoustically couple the sensor and the subject portion to each other.

Further, Japanese Patent Application Laid-open No. 2005-021380 discloses an apparatus that takes both a photoacoustic image and an ultrasound echo image.

Patent Literature 1: Japanese Patent Application Laid-open No. 2005-021380

Non Patent Literature 1: “Dedicated 3D Photoacoustic Breast Imaging” by Robert A. Kruger, Cherie M. Kuzmiak, Richard B. Lam, Daniel R. Reinecke, Stephen P. Del Rio, and Doreen Steed, Medical Physics 40, 113301 (2013)

### SUMMARY OF THE INVENTION

The apparatus described in Japanese Patent Application Laid-open No. 2005-021380 is capable of performing both photoacoustic imaging and ultrasound echo imaging but has a problem with its poor image quality since a field of view may not be substantially broadened in the photoacoustic imaging. In order to perform both the photoacoustic imaging and the ultrasound echo imaging with high quality, it is preferable to separately provide each of a sensor array in which a plurality of receiving elements for the photoacoustic imaging are arranged and an ultrasound echo imaging sensor array on the inner surface of a hemispherical shell-shaped array support unit. However, a method for arranging these two types of sensor arrays has not been satisfactorily discussed. In addition, when the two types of sensor arrays are used in combination, an array support unit for supporting these sensor arrays is likely to be upsized compared with a case in which only the receiving element array for the photoacoustic imaging is used. Further, when an array support unit is accommodated in a bed on which an examinee lays down like the apparatus described in “Dedicated 3D Photoacoustic Breast Imaging,” the entire apparatus including the bed is upsized. In this case, there is a likelihood that the posture of an examinee under testing is restricted due to the shape and the size of the apparatus, which in turn impairs the easiness of the examinee. In addition, since an excessive distance is generated between an assisting person and an examinee, there is a likelihood that the operability of an assisting operation by the assisting person is impaired.

The present invention has been made in view of the above problems. The present invention has an object of providing a technology for performing favorable imaging with an acoustic wave receiving apparatus that performs both photoacoustic imaging and ultrasound echo imaging.

The present invention provides an acoustic wave receiving apparatus comprising:

a light irradiation unit optically connected to a light source configured to irradiate a subject portion of an examinee with light;

a receiving array having a plurality of receiving elements configured to receive photoacoustic waves generated by an irradiation of the light from the light irradiation unit onto the subject portion and output photoacoustic signals;

a transmitting and receiving array having a plurality of transmitting and receiving elements configured to transmit ultrasound waves to the subject portion and receive echo waves reflected from the subject portion and then output ultrasound signals;

an array support unit having a first portion supporting the receiving array configured to direct a plurality of a reception directivity axes of the receiving elements toward an effective reception region and a second portion supporting the transmitting and receiving array;

a scanning unit configured to move the array support unit so as to scan the receiving array and the transmitting and the receiving array integrally with respect to the subject portion; and wherein

the array support unit is so configured that the effective reception region and a focusing region formed by the transmitting and receiving array do not overlap each other.

According to an embodiment of the present invention, it is possible to provide a technology for performing favorable imaging with an acoustic wave receiving apparatus that performs both photoacoustic imaging and ultrasound echo imaging.

Further features of the present invention will become apparent from the following description of exemplary embodiments with reference to the attached drawings.

### BRIEF DESCRIPTION OF THE DRAWINGS

FIGS. 1A to 1C are views showing a first acoustic wave receiving apparatus;

FIGS. 2A and 2B are views showing a second acoustic wave receiving apparatus;

FIGS. 3A and 3B are views showing a third acoustic wave receiving apparatus;

FIG. 4 is a graph showing the sensitivity characteristics of receiving elements;

FIG. 5 is a view showing the structure of an ultrasound echo linear array;

FIG. 6 is a view for describing electronic focusing; and

FIG. 7 is a view showing the connection between a computer and its peripheral devices.

### DESCRIPTION OF THE EMBODIMENTS

Hereinafter, a description will be given of a preferred embodiment of the present invention with reference to the drawings. However, sizes, materials, shapes, their relative arrangements, or the like of constituents that will be described below may be appropriately changed depending on the configurations or various conditions of apparatuses to which the present invention is applied. Accordingly, the sizes, materials, shapes, their relative arrangements, or the like of the constituents do not intend to limit the scope of the invention to the following description.

The present invention relates to a technology for detecting acoustic waves propagating from a subject portion and generating and acquiring characteristic information inside the subject portion. Accordingly, the present invention is grasped as a subject-portion information acquiring apparatus or its control method, or grasped as a subject-portion information acquiring method or a signal processing method. In addition, the present invention is grasped as a program that causes an information processing apparatus having hardware resources such as a CPU to perform these methods, or grasped as a storage medium storing the program. Moreover, the present invention is grasped as a photoacoustic-wave measuring apparatus or its control method.

The present invention is applicable to a subject-portion information acquiring apparatus using photoacoustic tomography that irradiates with light (electromagnetic waves) a subject portion and receives (detects) acoustic waves generated and propagated inside the subject portion or at the specific position of the surface of the subject portion according to a photoacoustic effect. Since such an apparatus acquires characteristic information inside a subject portion in the form of image data, characteristic distribution information, or the like based on photoacoustic measurement, it is called a photoacoustic imaging apparatus, a photoacoustic image forming apparatus, or simply a photoacoustic apparatus. Instead, since the apparatus of the present invention tests the inside of a subject portion, it may be called an acoustic wave receiving apparatus.

Characteristic information acquired by a photoacoustic apparatus includes the generation source distribution of acoustic waves generated by the irradiation of light, an initial sound pressure distribution inside a subject portion, a light energy absorption density distribution and an absorption coefficient distribution derived from an initial sound pressure distribution, the distribution of the concentration of substances constituting tissues, or the like. The concentration of substances includes oxygen saturation, oxyhemoglobin concentration, deoxyhemoglobin concentration, total hemoglobin concentration, or the like. The total hemoglobin concentration is the sum of the oxyhemoglobin concentration and the deoxyhemoglobin concentration. In addition, fat, collagen, a water distribution, or the like is also targeted. Moreover, the characteristic information may be calculated as distribution information on respective positions inside a subject portion rather than being calculated as numerical data. That is, distribution information such as an absorption coefficient distribution and an oxygen saturation distribution may be handled as subject-portion information.

The present invention is also applicable to an apparatus using an ultrasound echo technology that transmits ultrasound waves to a subject portion, receives reflected waves (echo waves) reflected inside the subject portion, and acquires subject-portion information as image data. In the case of an apparatus using the ultrasound echo technology, acquired subject-portion information is information reflecting a difference in the acoustic impedance between tissues inside a subject portion.

In the present invention, acoustic waves are typically ultrasound waves and include elastic waves called sound waves or acoustic waves. Acoustic waves generated by a photoacoustic effect are called photoacoustic waves or light ultrasound waves, and also include reflected waves (echo waves) generated by the above ultrasound echo technology. Electrical signals converted from acoustic waves by a probe are also called acoustic signals, acoustic signals derived from photoacoustic waves are particularly called photoacoustic signals, and acoustic signals derived from reflected waves by the ultrasound echo technology are particularly called ultrasound signals. Note that ultrasound waves or acoustic waves that will be described in the specification do not intend to limit the wavelengths of their elastic waves.

As a subject portion in the present invention, the breast of a living body may be assumed. However, the subject portion is not limited to the breast, and other portions of the living body or a non-living-body material may be tested. Accordingly, the present invention is also grasped as a subject-portion information acquiring apparatus or its control method, or grasped as an acoustic wave receiving apparatus or its control method.

When a sensor array (receiving array) in which a plurality of receiving elements are arranged on the inner surface of a photoacoustic hemispherical shell-shaped array support unit and a linear sensor array (transmitting and receiving array) for ultrasound echo diagnosis are used in combination in an acoustic wave receiving apparatus, it becomes possible to perform imaging with both modalities. However, when both the sensor arrays are so arranged that their reception regions do not overlap each other, the member of the array support unit is upsized. In addition, when an image is taken in a wide range, the relative positional relationship between a subject portion and the array support unit on which the sensor array is mounted is changed by a scanning unit. However, when both the sensor arrays are separately arranged and the scanning unit is provided in each of the sensor arrays, the structure becomes complicated due to the upsized members of the scanning units. In addition, the moving distance of the array support unit is increased. As a result, an increase in cost and a lengthened image taking time are caused.

Further, there is a case that a support member for supporting an examinee is provided in an acoustic wave receiving apparatus. However, when the member of an array support unit is upsized, the support member is also upsized. As a result, the operability of an assisting person who assists an examinee is reduced. In addition, an examinee has a difficulty in taking an easy posture. Accordingly, in an acoustic wave receiving apparatus including both a photoacoustic imaging sensor array and an ultrasound echo imaging sensor array, it is necessary to downsize members and enhance the operability of an assisting person or the comfort of an examinee.

### Embodiment

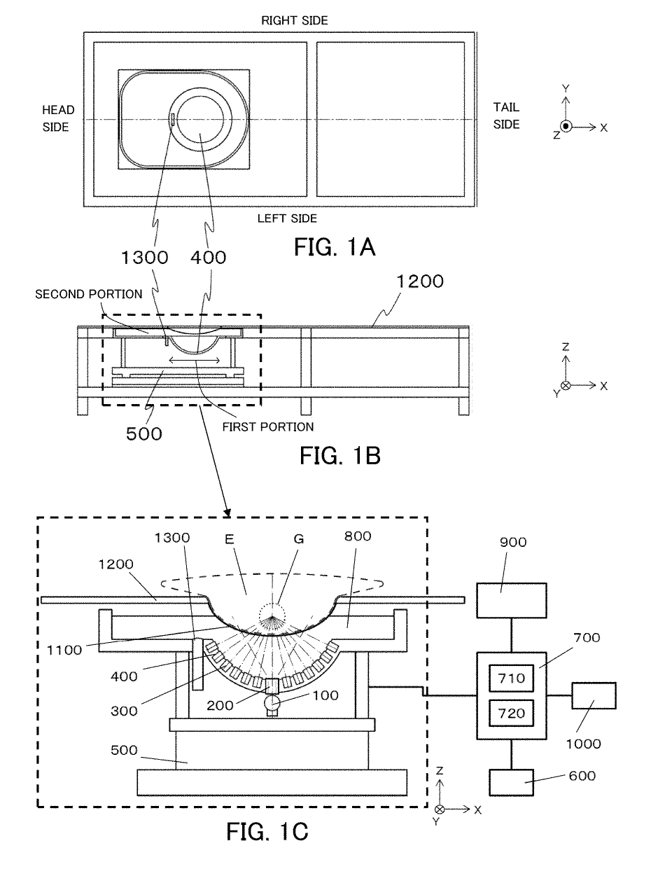

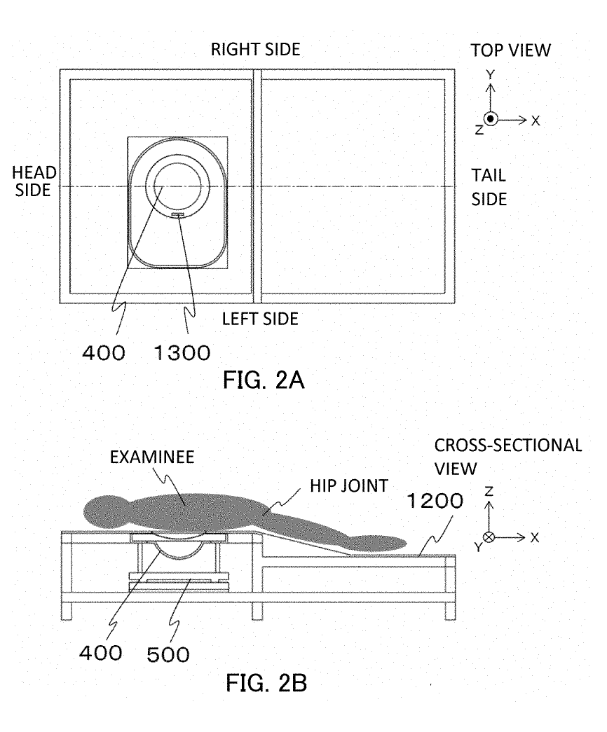

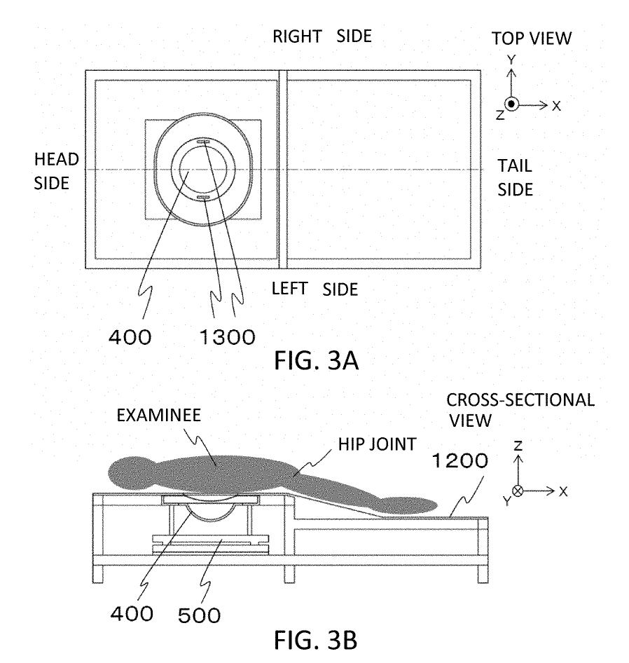

FIGS. 1A to 1C, FIGS. 2A and 2B, and FIGS. 3A and 3B are schematic views each showing the configuration and operation of an acoustic wave receiving apparatus according to an embodiment. The acoustic wave receiving apparatus acquires characteristic information on a subject portion E by a photoacoustic effect and an ultrasound echo technology. FIG. 1A is a top view of a first acoustic wave receiving apparatus, FIG. 1B is a cross-sectional view of a side surface thereof, and FIG. 1C is a view showing the details of a part surrounded by dashed lines in FIG. 1B. FIG. 2A is a top view of a second acoustic wave receiving apparatus, and FIG. 2B is a cross-sectional view of a side surface thereof. FIG. 3A is a top view of a third acoustic wave receiving apparatus, and FIG. 3B is a cross-sectional view of a side surface thereof.

(Basic Configuration)

A description will be given, with reference to FIG. 1C, of the basic configuration of a subject-portion information acquiring apparatus serving as the acoustic wave receiving apparatus of the embodiment. The acoustic wave receiving apparatus includes a light source 100, an optical system 200, a receiving array 300 including a plurality of receiving elements 310, a transmitting and receiving array 1300 including a plurality of transmitting and receiving elements 1310, an array support unit 400, a scanner 500, a subject-portion appearance information acquiring unit 600, a computer 700, a display 900, an inputting unit 1000, a shape maintaining portion 1100, and a support member 1200.

(Subject Portion)

The subject portion E is a subject to be measured. Specific examples of the subject portion E include a living body such as a breast and a phantom that is used to adjust or calibrate an apparatus and simulates the acoustic characteristics and optical characteristics of a living body. The acoustic characteristics specifically include the propagating speed and attenuation rate of acoustic waves. The optical characteristics specifically include the absorption coefficient and scattering coefficient of light. Inside or on the surface of the subject portion, light absorbers having a large light absorption coefficient exist. In a living body, the light absorbers include hemoglobin, water, melanin, collagen, fat, or the like. The phantom includes substances simulating optical characteristics as the light absorbers. Note that in FIG. 1C, the subject portion E is indicated by dashed lines for the sake of convenience.

(Light Source)

The light source 100 emits pulsed light. A laser is preferably used as the light source to obtain a large output, but a light emitting diode or the like may be used instead. In order to effectively generate photoacoustic waves, it is necessary to emit light in a substantially short period of time according to the heat characteristics of a subject portion. When the subject portion is a living body, the pulse width of the pulsed light emitted from the light source 100 is preferably several tens of nanoseconds or less. In addition, the wavelength of the pulsed light falls within a near-infrared region called the window of a living body and is preferably in the range of about 700 nm to 1200 nm. Since the light falling within this region reaches relatively the deep part of a living body, information on the deep part may be acquired. When only the surface part of a living body is to be measured, light falling within a region ranging from the region of visible light having a wavelength of about 500 nm to 700 nm to the near-infrared region may be used. In addition, the wavelength of the pulsed light preferably has a high absorption coefficient with respect to a subject to be observed.

(Optical System)

The optical system 200 guides the pulsed light generated from the light source 100 to the subject portion E. The optical system 200 specifically includes an optical device such as a lens, a mirror, a prism, an optical fiber, and a diffusion plate, or their combinations. In addition, it is preferable to change, when the light is guided, the shape or density of the light using these optical devices so that the irradiated light has a desired light distribution. In the embodiment, the optical system 200 is configured to illuminate a region at the center of the curvature of a hemisphere. The optical system 200 corresponds to the light irradiation unit of the present invention.

In addition, a maximum permissible exposure (MPE) has been set according to safety standards as for the intensity of light permitted to be irradiated onto living-body tissues. As the safety standards, “IEC 60825-1: Safety of laser products” has been, for example, known. In addition, “JIS C 6802: Safety of laser products,” “FDA: 21CFR Part 1040.10,” “ANSI 2136.1: Laser safety standards,” or the like has been known. The maximum permissible exposure stipulates the intensity of light permitted to be irradiated per unit area. Therefore, much light may be guided to the subject portion E by collectively irradiating with the light a large area of the surface of the subject portion E. As a result, photoacoustic waves may be received at a high SN ratio. Therefore, instead of focusing the light with a lens, it is more preferable to spread the light to a certain degree of area as indicated by two-dot chain lines in FIG. 1C.

(Receiving Elements)

The receiving elements 310 are elements that receive photoacoustic waves and convert the same into electrical signals. The receiving array 300 includes the plurality of receiving elements 310. The receiving elements are preferably those that have high receiving sensitivity and a wide frequency band with respect to photoacoustic waves from the subject portion E. Note that the name of “receiving elements” does not intend to limit the material or the like of the elements. As the receiving elements 310, various elements generally used to receive acoustic waves (ultrasound waves) may be used. The acoustic wave receiving apparatus includes a signal processing circuit that applies amplifying processing or digital converting processing to the electrical signals converted by the receiving elements 310.

As the material of the receiving elements 310, a piezoelectric ceramic material represented by lead zirconate titanate (PZT), a polymer piezoelectric film material represented by polyvinylidene difluoride (PVDF), or the like may be used. In addition, members made of a material other than a piezoelectric material, e.g., capacitive elements such as capacitive micro-machined ultrasonic transducers (cMUT), receiving elements using a Fabry-Perot interferometer may be used. Moreover, since the cMUT has a wider frequency band compared with piezoelectric elements whose frequency band is determined based on the thickness of a piezoelectric material, it is suitable for receiving photoacoustic waves having an unknown frequency band generated from a subject portion.

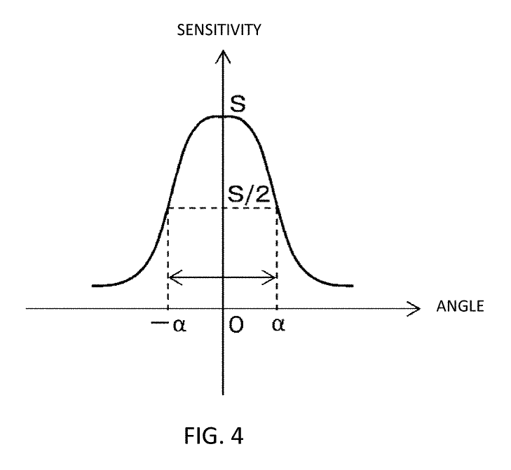

FIG. 4 is a graph showing an example of the receiving sensitivity characteristics of the receiving elements 310. In FIG. 4, “angles” indicated by a horizontal axis are incident angles formed between the normal directions of the receiving surfaces of the receiving elements 310 and the incident direction of photoacoustic waves. A vertical axis indicates the relative values of receiving sensitivity at the respective incident angles. In FIG. 4, the receiving sensitivity obtained when the acoustic waves are incident from the normal directions of the receiving surfaces is the highest. That is, when the incident angle is zero, the sensitivity becomes S (maximum value). Further, the receiving sensitivity reduces as the incident angles increase. It is assumed that the receiving elements 310 according to the embodiment have a circular plan-shaped receiving surface.

In addition, it is assumed that the incident angle at which the receiving sensitivity becomes 50% (S/2) of the maximum value is α. In the present invention and the present specification, it is assumed that a region in which the photoacoustic waves are incident on the receiving surfaces of the receiving elements 310 at an incident angle of α or less is the reception region of the elements and that a direction in which the absolute values of angles formed with the normal directions of the receiving surfaces are α or less (a direction in which the receiving sensitivity is half or more of the maximum value) is called a receiving direction. However, the reception region is not limited to such a half-value width but may be set according to element characteristics, accuracy needed for measurement, or the like. In FIG. 1C, directions in which the receiving sensitivity of the receiving elements 310 is the highest are indicated by chain lines.

(Transmitting and Receiving Elements)

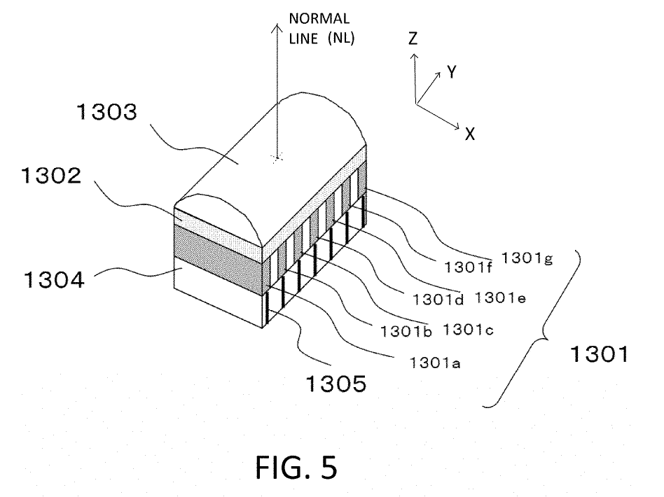

The transmitting and receiving elements 1310 are elements that receive reflected waves and convert the same into electrical signals while transmitting ultrasound waves. The transmitting and receiving array 1300 includes the plurality of transmitting and receiving elements 1310. Note that the name of “transmitting and receiving elements” does not intend to limit the wavelength of transmitting waves or echo waves. FIG. 5 shows the structure of a linear sensor array as an example of the transmitting and receiving array including the plurality of transmitting and receiving elements, the transmitting and receiving array constituting the acoustic wave receiving apparatus of the embodiment. Reference numerals 1301, 1302, 1303, 1304, and 1305 denote a piezoelectric element array, an acoustic matching layer, an acoustic lens, a backing material, and a lead wire, respectively. The acoustic wave receiving apparatus includes signal processing circuits that apply amplifying processing or digital converting processing to electrical signals converted by the transmitting and receiving elements 1310. These signal processing circuits may be shared with the receiving elements 310. A configuration in which a group of receiving elements is arranged on a hemispherical array support unit and a group of transmitting and receiving elements is arranged in a linear array shape like the present apparatus is preferably used in for, for example, an acoustic wave receiving apparatus that primarily performs photoacoustic imaging and secondarily performs ultrasound echo imaging.

The piezoelectric element array 1301 is one in which a plurality of strip-shaped piezoelectric elements 1301a to 1301g are arrayed in a linear shape. According to this configuration, the transmitting and receiving elements 1310 of the present invention are constituted in a linear array shape. The respective piezoelectric elements are connected to lead wires 1305a to 1305g. In addition, the acoustic matching layer 1302 is provided on the surface of the piezoelectric element array 1301 from which ultrasound waves are to be transmitted. On the other hand, the backing material 1304 is provided on the surface of the piezoelectric element array 1301 on the side opposite to the side from which ultrasound waves are to be transmitted. Moreover, the acoustic lens 1303 (acoustic-waves focusing means) is provided on the surface of the acoustic matching layer 1302 on the side opposite to the side of the piezoelectric element array 1301.

The piezoelectric elements 1301a to 1301g constituting the piezoelectric element array 1301 may be replaced by the same type of piezoelectric elements as the above receiving elements 310. Note that piezoelectric elements made of a piezoelectric ceramic material or a polymer piezoelectric film material are particularly preferable as the transmitting and receiving elements 1310 since they are suitable for transmitting ultrasound waves.

As the acoustic lens 1303, a cylindrical lens that focuses ultrasound waves in a direction orthogonal to the arraying direction of the piezoelectric element array 1301 is used. By setting a region in which ultrasound waves are to be focused by the cylindrical lens in the region of the interest of a subject portion, it is possible to selectively receive reflected waves generated from the region of the interest of the subject portion E while selectively transmitting the ultrasound waves to the region of the interest of the subject portion. As a material constituting the acoustic lens, a material having acoustic characteristics similar to those of living-body tissues is preferably used. An example of such a material, silicon rubber has been known. When the acoustic lens is constituted by a material having sound speed slower than that inside living-body tissues, the acoustic lens is formed to have a convex shape, a focal position is determined by the curvature of a convex surface, and a focusing size is determined by a focal distance and the width of the cylindrical lens. On the other hand, as a method for focusing ultrasound waves in the arraying direction of the piezoelectric elements 1301, electronic focusing is used. The electronic focusing (acoustic-waves focusing means) will be described later.

The acoustic matching layer 1302 is provided to efficiently transmit acoustic signals. Generally, the acoustic impedance of a piezoelectric element material is greatly different from that of a living body or an acoustic matching fluid. Therefore, when the piezoelectric element material is brought into direct contact with the acoustic matching fluid, it is not possible to efficiently transmit acoustic signals due to large reflection at the interface. In view of this, the acoustic matching layer 1302 constituted by a substance having intermediate acoustic impedance is inserted between a piezoelectric element material and a living body or an acoustic matching fluid to efficiently transmit acoustic signals. As an example of a material constituting the acoustic matching layer 1302, an epoxy resin, quartz glass, or the like has been known.

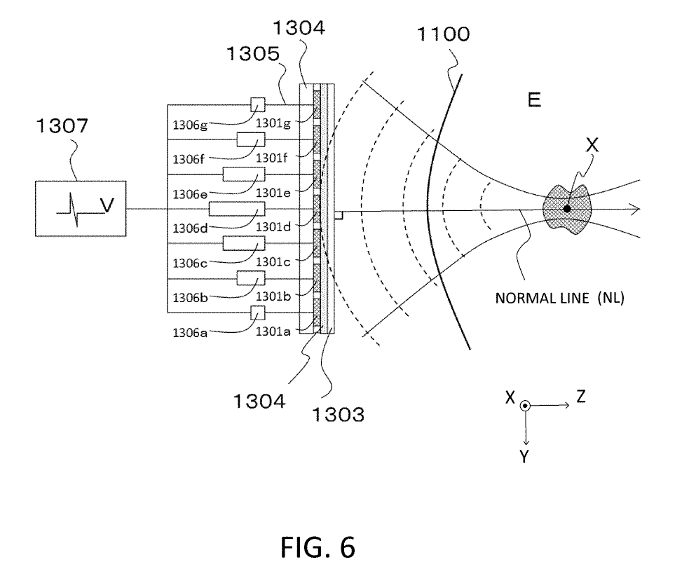

Next, a description will be given, with reference to FIG. 6, of the electronic focusing using a linear array probe. Reference numeral 1306 denotes a variable delay element, and reference numeral 1307 denotes a pulser receiver. Variable delay elements 1306a to 1306g are connected to the piezoelectric elements 1301a to 1301g arranged in plurality, respectively, via the lead wires 1305. The respective variable delay elements 1306a to 1306g are connected to the pulser receiver 1307.

The variable delay elements 1306 are constituted by long and narrow electric wires wound in a coil shape or the like and serve to delay the conduction of electrical signals transmitted through the electric wires. In addition, the variable delay elements 1306 are capable of adjusting the delay times of electrical signals by changing a plurality of taps provided at the middle of the coils. The pulser receiver 1307 is a unit that receives acoustic signals converted into voltages by the piezoelectric element array 1301. Acoustic waves generated from a position X of an acoustic-wave generation source reach the piezoelectric element array 1301 with respective time differences τa, τb, τC, τd, τe, τf, and τg. Note that the time differences may be time differences with respect to moments at which acoustic waves are generated, time differences with respect to moments at which the acoustic waves are generated in any of the piezoelectric elements included in the piezoelectric element array 1301, or time differences with respect to reference timings set separately from such time differences.

Acoustic waves having reached the piezoelectric element array 1301 are converted into voltages (electrical signals). The electrical signals are received by the pulser receiver 1307 via the variable delay elements 1306. At this time, the variable delay elements 1306 correct the differences between the position X of the acoustic-wave generation source and the respective piezoelectric elements 1301 to make the phases of the respective electrical signals coincide with each other. In the example of FIG. 6, piezoelectric elements closer to the position X are given a longer delay time by the corresponding variable delay elements 1306. In addition, when piezoelectric elements at an equal distance from the position X are given the same delay time by the corresponding variable delay elements 1306 (τa=τg<τb=τf<τc=τe<τd), the phases of the electrical signals at a time at which electrical signals are received by the pulser receiver 1307 coincide with each other. Thus, the electronic focusing of the linear array probe is performed by controlling delay times to be given by the variable delay elements 1306. As a result, it is possible to control a focal distance or a focal position in the arraying direction of the piezoelectric elements 1301.

When a command pulse for transmitting ultrasound waves to the region of the interest of the subject portion E is transmitted from the pulser receiver 1307, the pulse reaches the piezoelectric elements 1301a to 1301g. At this time, times at which the pulse reaches the respective elements are delayed by the variable delay elements 1306a to 1306g. Specifically, the pulse reaches the piezoelectric elements 1301a to 1301g with the time differences τPa, τPb, τPc, τPd, τPe, τPf, and τPg, respectively. Thus, by adjusting timings at which ultrasound waves are generated from the respective elements according to the distances between the respective piezoelectric elements 1301a to 1301g and the region of the interest of the subject portion E as described above, the transmitted ultrasound waves are focused on the region of the interest of the subject portion E.

When reflected waves generated from the region of the interest of the subject portion E are received, signal delaying processing is performed in the same manner as the above according to the distances between the region of the interest and the respective elements. The reflected waves generated from the region of the interest of the subject portion E reach the piezoelectric elements 1301a to 1301g with time differences τRa, τRb, τRc, τRd, τRe, τRf, and τRg, respectively, and then are converted into electrical signals. The respective electrical signals are delayed by the corresponding variable delay elements 1306a to 1306g by the times τRa to τRg and then received by the pulser receiver 1307.

At this time, the respective variable delay elements 1306 correct times according to the distances between the respective piezoelectric elements and the region of the interest of the subject portion E to make phases at which the electrical signals are received by the pulser receiver 1307 coincide with each other. As a result, a focusing region is generated at the region of the interest of the subject portion E, and reflected waves generated from the focusing region are selectively received. In the example of FIG. 6, the delay times are made longer as the distances between the region of the interest and the piezoelectric elements are shorter.

Forming the focusing region of electrically transmitted/received ultrasound waves as described above is called the electronic focusing. The acoustic wave receiving apparatus of the present invention makes the depth of a focusing region formed at the region of the interest of the subject portion E by the electronic focusing the same as the depth of a focusing region to be formed by the acoustic lens 1303 described above. Here, the “depth” represents a distance from a surface at which the relative position of a group of transmitting and receiving elements in a linear array shape or a group of receiving elements arranged in a hemispherical shape changes with respect to the subject portion, or represents a distance from the supporting surface of the support member. It is assumed that the depth is the same when a position in a z-axis direction in the figures is the same.

(Array Support Unit)

The array support unit 400 is a container having a shape in which a hemispherical portion (first portion) and a substantially bottomed cylindrical portion (second portion) are combined together. The hemispherical portion is arranged so as to protrude from a cylindrical bottom surface forming the second portion. With the first and second portions combined together, the array support unit 400 is allowed to accommodate an acoustic matching fluid 800 as a whole. The plurality of receiving elements 310 are provided on the inner surface of the hemisphere. That is, the array support unit 400 supporting the plurality of receiving elements 310 constitutes the receiving array 300. The plurality of transmitting and receiving elements 1310 are provided at a part of a region corresponding to the bottom surface of the second portion outside the hemisphere. In addition, the optical system 200 is provided at the bottom part (pole) of the hemisphere. The acoustic matching fluid 800 is filled inside the array support unit 400. The array support unit 400 is preferably made of a metal material or the like having mechanical strength to support these members.

In addition, since attenuation is caused when ultrasound waves propagate through the acoustic matching fluid, it is preferable to shorten the distance between a subject portion and the transmitting and receiving elements 1310. Therefore, in the acoustic wave receiving apparatus of the embodiment, the transmitting and receiving elements 1310 are provided on the bottom surface of the substantially cylindrical portion of the array support unit 400.

Each of the centers in a receiving direction (highest sensitivity direction) of the plurality of receiving elements 310 provided on the array support unit 400 is oriented to the center of the curvature of the hemisphere. The centers in the receiving direction are indicated by chain lines focusing on a partial region inside the subject portion E. FIG. 1C is a cross-sectional view obtained when the hemispherical array support unit 400 is cut off at it central axis. As described above, each of the plurality of receiving elements 310 is arranged on the array support unit 400 so as to be capable of receiving photoacoustic waves generated at a specific region with high sensitivity. In the embodiment, this specific region is called an effective reception region. In other words, the effective reception region indicates an isocenter at where the plurality of the reception directivity axes of the receiving elements intersect commonly.

In a case in which the plurality of receiving elements 310 are arranged as described above, the resolution of subject-portion information obtained using a receiving signal according to a method that will be described later becomes higher at the center of the curvature of the hemisphere and becomes lower with a distance from the center. In the embodiment, the effective reception region represents a region from the point of the highest resolution (the center of the curvature of the hemisphere) to the point of resolution half the highest resolution, and corresponds to a region G surrounded by dotted lines in FIG. 1C.

In the acoustic wave receiving apparatus of the present invention, the depth of a focusing region formed at the region of the interest of a subject portion by the transmitting and receiving elements that receive an ultrasound echo image is positioned within a range (at or below the upper end, and at or above the lower end of the region indicated by reference symbol G in the z-axis direction) in the depth direction of the region G at which a photoacoustic image is acquired with high sensitivity.

Thus, it is possible to take a photoacoustic image and an ultrasound echo image at the same depth in a subject portion with high quality. In addition, in the acoustic wave receiving apparatus of the present invention, the plurality of transmitting and receiving elements 1310 are arranged outside the reception region of the plurality of receiving elements 310, and the plurality of receiving elements 310 are arranged outside the transmission and reception region of the plurality of transmitting and receiving elements 1310.

Note that the highest sensitivity directions of the respective receiving elements may not necessarily cross each other in the first portion so long as a desired effective reception region is formed. In addition, the each of the highest sensitivity direction (reception directivity axis) of at least some of the plurality of receiving elements 310 supported by the array support unit 400 may be so oriented to a specific region that photoacoustic waves generated at a specific region are received with high sensitivity. Provided that this condition is satisfied, the shape of the first portion is not limited to a hemisphere. For example, the first portion may have a spherical crown shape, a shape obtained by cutting off a part of an ellipsoid, or a shape obtained by combining a plurality of plane surfaces or curved surfaces together.

In addition, the shape of the second portion is not limited to a substantially bottomed cylindrical shape. For example, a member including a bottom surface part and a side surface part may be used as the second portion, the bottom surface part being connected to the first portion and allowing the transmitting and receiving elements to be provided thereon, the side surface part being allowed to accommodate the acoustic matching fluid of an amount necessary to maintain the acoustic matching between a subject portion and the piezoelectric element array. In addition, the side surface part of the second portion is not a shape steep from the bottom surface part but may be connected to the bottom surface part in a curved shape. For example, as the second portion, FIGS. 1A to 1C show a shape in which a cylinder and a cuboid with its corners rounded are combined together, and FIGS. 3A and 3B show a substantially elliptic cylindrical shape.

(Scanner)

The scanner 500 is a unit that moves the array support unit 400 in the X and Y directions of FIG. 1C to change the relative position of the array support unit 400 with respect to the subject portion E. Therefore, the scanner 500 includes a guiding mechanism in the X and Y directions, a driving mechanism in the X and Y directions, and a position sensor that detects positions in the X and Y directions of the array support unit 400 (each not shown). Since the array support unit 400 is mounted on the scanner 500, a linear guide or the like capable of withstanding a large load is preferably used as the guiding mechanism. In addition, a lead screw mechanism, a link mechanism, a gear mechanism, a hydraulic mechanism, or the like may be used as the driving mechanism. In order to generate a driving force, a motor or the like may be used. Moreover, a potentiometer or the like using an encoder, a variable resistor, or the like may be used as the position sensor. Note that any member may be used as the scanner 500 so long as the member is capable of moving the array support unit 400 with respect to the subject portion E. The scanner 500 corresponds to the scanning unit of the present invention.

The acoustic wave receiving apparatus of the present invention is so configured that the effective reception region to be formed by the plurality of receiving elements 310 and the focusing region to be formed by the transmitting and receiving elements 1310 do not overlap each other. The paths of photoacoustic signals received by the plurality of receiving elements 310 and the transmitting and receiving elements 1310 do not preferably overlap each other. In order to prevent the paths of the photoacoustic signals and the transmitting and receiving elements 1310 from overlapping each other, the transmitting and receiving elements 1310 may be arranged outside the reception regions (regions having surfaces at which the photoacoustic signals are received at an angle of −α or less or an angle of α or more) of the receiving elements 310. Therefore, the hemispherical portion for forming the effective reception region G described above and the transmitting and receiving elements 1310 are arranged to offset in a horizontal direction (XY direction). Therefore, the bottom of the second portion of the array support unit 400 has a shape suitable for arranging the transmitting and receiving elements 1310. As described above, since the acoustic wave receiving apparatus of the present invention is so configured that the effective reception region to be formed by the plurality of receiving elements 310 and the focusing region to be formed by the transmitting and receiving elements 1310 do not overlap each other, influence on photoacoustic signals from a subject portion by the transmitting and receiving elements when acquiring the photoacoustic signals may be reduced. Similarly, influence on ultrasound signals from a subject portion by the receiving elements 310 when acquiring the ultrasound signals may be reduced.

Referring FIGS. 5 and 6, the transmitting and receiving elements 1310 (the transmitting and receiving array) has a common receiving surface. The focusing region of the transmitting and receiving elements 1310 is typically substituted a central normal line NL of the common receiving surface (1303) therein.

In other words, the acoustic wave receiving apparatus of the present invention is so configured that the effective reception region to be formed by the plurality of receiving elements 310 and a central normal line NL of the receiving surface 1303 of the transmitting and receiving elements 1310 do not overlap each other.

In addition, in order to obtain a fine image of the region of the interest of the subject portion E with both ultrasound echo imaging and photoacoustic imaging, each of the effective reception region G and the focusing region of the transmitting and receiving elements 1310 needs to overlap with the region of the interest. The scanner 500 is configured so as to realize a moving distance or a moving position satisfying this condition.

(Subject-Portion Appearance Information Acquiring Unit)

The subject-portion appearance information acquiring unit 600 is a unit that acquires appearance information on the subject portion E. As the subject-portion appearance information acquiring unit 600, an imaging apparatus such as a camera that takes an image of the subject portion E may be, for example, used. A taken image of the subject portion E may be displayed on the display 900 that will be described later. In addition, the operator of the acoustic wave receiving apparatus may specify the region of the interest of the subject portion E to be tested by the inputting unit 1000 after confirming a taken image displayed on the display 900. Moreover, for image reconfiguration, acquired appearance information may be used to acquire a light amount distribution or an acoustic-wave attenuation degree inside the subject portion E.

(Computer)

The computer 700 has a calculating unit 710 and a storing unit 720. The calculating unit 710 is typically constituted by an element such as a CPU, a GPU, an A/D converter, and an amplifier and a circuit such as a FPGA and an ASIC. Note that the calculating unit may be constituted not only by one element and one circuit but also by a plurality of elements and a plurality of circuits. In addition, respective processing to be performed by the computer 700 may be performed by any of the elements and the circuits. The storing unit 720 is typically constituted by a storing medium such as a ROM, a RAM, and a hard disk. Note that the storing unit may be constituted not only by one storage medium but also by a plurality of storing media.

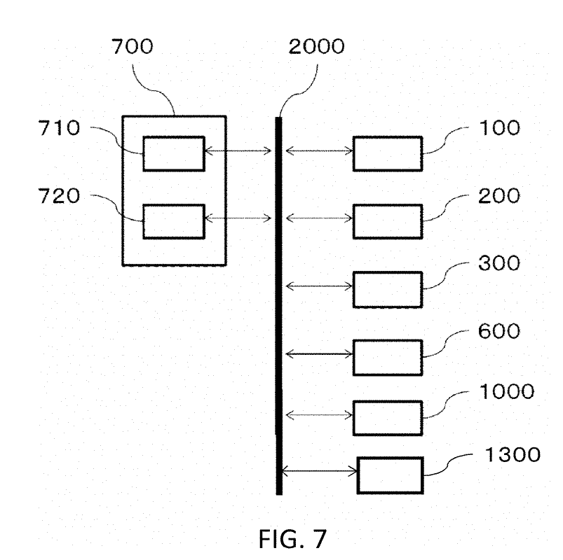

The calculating unit 710 applies signal processing to electrical signals output from the plurality of receiving elements 310 and the transmitting and receiving elements 1310. In addition, the calculating unit 710 serving as a controlling unit controls the operations of the respective configurations constituting the subject-portion information acquiring apparatus via a bus 2000 as shown in FIG. 7. An operation to be controlled by the calculating unit 710 includes the switching of an imaging mode between photoacoustic imaging and ultrasound echo imaging, the specifying and changing of an imaging region, or the like. Moreover, the computer 700 is preferably configured to be capable of simultaneously performing the pipeline processing of a plurality of signals. Thus, it is possible to shorten a time for acquiring subject-portion information. The respective processing to be performed by the computer 700 may be stored in advance in the storing unit 720 as programs to be executed by the calculating unit 710.

In the switching of the imaging mode described above, the ultrasound echo imaging may be performed after the photoacoustic imaging, or the photoacoustic imaging may be performed after the ultrasound echo imaging. In addition, the photoacoustic imaging and the ultrasound echo imaging may be alternately performed. When images of the same region of the subject portion E are taken by the photoacoustic imaging and the ultrasound echo imaging and displayed in an overlapped manner, the photoacoustic imaging and ultrasound echo imaging are preferably alternately performed to reduce the positional deviation between both the photoacoustic imaging and the ultrasound echo imaging. Alternatively, it may be possible to display a photoacoustic image on the display 900 after performing the photoacoustic imaging (or the ultrasound echo imaging) of the entire subject portion E, specify the region of the interest of the displayed image with the inputting unit 1000, and perform the ultrasound echo imaging (or the photoacoustic imaging) of the region of the interest. The entire subject portion E is screened by the photoacoustic imaging (or the ultrasound echo imaging), and then the ultrasound echo imaging (or the photoacoustic imaging) is performed based on the region of interest. Therefore, since there is no need to perform both the imaging modes on the entire subject portion E, it is possible to shorten a testing time.

The calculating unit 710 is capable of generating photoacoustic image data derived from electrical signals (photoacoustic signals) output from the receiving elements 310 and ultrasound image data derived from electrical signals (ultrasound signals) output from the transmitting and receiving elements 1310. In order to generate the image data, any known image reconstruction method (such as phasing addition, filtered back projection, universal back projection, Fourier transform, and inverse problem analysis) may be used. At this time, the computer 700 serves as the information processing unit of the present invention. Different methods may be separately used between the photoacoustic image data and the ultrasound image data. The generated photoacoustic image data or the ultrasound image data may be displayed on the display 900 or may be stored in a memory.

(Acoustic Matching Fluid)

The acoustic matching fluid 800 is filled in the space between the subject portion E and the receiving elements 310 and acoustically couples the subject portion E and the receiving elements 310 to each other. Therefore, the acoustic matching fluid 800 is preferably arranged between the receiving elements 310 and the shape maintaining portion 1100 and between the shape maintaining portion 1100 and the subject portion E. In addition, the acoustic matching fluid 800 arranged between the receiving elements 310 and the shape maintaining portion 1100 may be different from the acoustic matching fluid 800 arranged between the shape maintaining portion 1100 and the subject portion E.

The acoustic matching fluid 800 is preferably a material having acoustic impedance closer to those of the subject portion E and the receiving elements 310. In addition, the acoustic matching fluid 800 is more preferably a material having intermediate acoustic impedance between the impedance of the subject portion E and the impedance of the receiving elements 310. Moreover, the acoustic matching fluid 800 is preferably a material that allows pulsed light generated from the light source 100 to pass through. Further, the acoustic matching fluid 800 is preferably a liquid. Specifically, as the acoustic matching fluid 800, a liquid such as water and ricinus oil, a gel, or the like may be used.

(Display)

The display 900 (display unit) displays subject-portion information output from the computer 700 as a distributed image, numerical data on a specific interest region, or the like. As the display, any type of display such as a liquid crystal display and an organic EL display may be used. Note that the display 900 may be provided separately from the acoustic wave receiving apparatus of the present invention.

(Inputting Unit)

The inputting unit 1000 is a user interface for allowing a user to input and specify desired information to the computer 700. As the inputting unit 1000, a keyboard, a mouse, a touch panel, a dial, a bottom, or the like may be used. When a touch panel is employed as the inputting unit 1000, the display 900 may be the touch panel serving also as the inputting unit 1000.

(Shape Maintaining Unit)

The shape maintaining portion 1100 is a member for maintaining the shape of the subject portion E. The shape maintaining portion 1100 is attached to the support member 1200. In order to change the maintained shape of the subject portion E or correspond to a difference in the size of the subject portion E between individuals, it is preferable to provide a configuration in which a plurality of shape maintaining portions having different shapes and sizes are replaceable.

When the subject portion E is a breast, the shape maintaining portion 1100 is preferably formed into a spherical crown shape or a bowl shape in order to reduce the deformation of the shape of the breast. Note that the shape of the shape maintaining portion 1100 may be designed according to the volume of the subject portion or the desired shape of the subject portion after being held. When light is irradiated onto the subject portion E via the shape maintaining portion 1100, the shape maintaining portion 1100 preferably allows the irradiated light to pass through. Therefore, as the material of the shape maintaining portion 1100, polymethylpentene, polyethylene terephthalate, or the like is preferably used.

In addition, as other materials of the shape maintaining portion 1100, a material having flexibility such rubber deformable corresponding to the shape of the subject portion E may be used. The material having flexibility is advantageous in that it is hardly wrinkled when the subject portion E is held. Moreover, a member allowing light from the light source 100 to pass through at high transmittance (preferably a transmittance of 90% or more) is preferably used. Specifically, silicon rubber, urethane rubber, styrene elastomer, olefin elastomer, acrylic elastomer, or the like is preferably used.

(Support Member)

The support member 1200 supports an examinee and includes an insertion opening through which the subject portion of the examinee is to be inserted. In addition, as shown in FIG. 1C, the array support unit 400, the scanner 500, or the like is accommodated under the support member 1200. The support member 1200 shown in FIG. 1C is suitable for supporting an examinee in a prone position. As a subject portion, a breast is assumed. The array support unit 400 to be moved by the scanner 500 of the acoustic wave receiving apparatus of the present invention is a relatively large member having the hemispherical portion and the bottom portion on which the transmitting and receiving array may be arranged. Therefore, the size of the support member 1200 becomes also relatively large. As a result, the operability of an assisting person or the comfort of an examinee may be influenced.

According to studies by the inventors, it has been found that the width (distance in the Y direction) of the support member 1200 needs to fall within a preferred range in order to allow an examinee to get on the support member 1200 without a sense of uneasiness and an assisting person to favorably perform the operation of assisting the examinee. In addition, as shown in FIGS. 2B and 3B, it has been found that it is easy for an examinee to make his/her region from a hip joint to legs slightly lowered to support the entire body. Moreover, it has been found that it is preferable to provide a member for adjusting a position at which a hip joint is lowered on the support member 1200 since there is a difference in the body size between individuals.

(First Arrangement)

Hereinafter, a description will be given of the arrangement and the configuration of the transmitting and receiving elements 1310 by which an assisting person is allowed to favorably perform an operation and an examinee is allowed to take an easy posture. In the first acoustic wave receiving apparatus of FIGS. 1A to 1C, a positional relationship in an X-axis direction is defined as follows. That is, a direction in which the head of an examinee is arranged is called a head side, and a direction in which the legs of the examinee are arranged is called a tail side. Here, in the case of, for example, FIGS. 1A to 1C, the transmitting and receiving elements 1310 are arranged at a place closer to the head side than the receiving elements 310. In addition, the transmitting and receiving elements 1310 are arranged at a place relatively closer to the head side in the array support unit 400.

In the above configuration in which the transmitting and receiving elements 1310 are arranged on the head side of the array support unit 400, the support member 1200 is expanded to the head side compared with a configuration in which a simple hemispherical array support unit is provided but is not expanded to right and left sides. Therefore, an assisting person is allowed to favorably perform the operation of assisting an examinee from the right and left sides of the support member 1200. By this configuration, assisting operability is not reduced compared with an acoustic wave receiving apparatus including a simple hemispherical array support unit 400.

Note that even in a configuration in which the transmitting and receiving elements 1310 are arranged on the tail side of the array support unit 400, an assisting person is allowed to favorably perform the operation of assisting an examinee from the right and left sides of the support member 1200. However, as will be described later, the transmitting and receiving elements 1310 are preferably arranged on the head side in view of making an examinee take a comfortable posture.

(Second Arrangement)

In the second acoustic wave receiving apparatus of FIGS. 2A and 2B, the transmitting and receiving elements 1310 are arranged on one of the right and left sides of the array support unit 400 when seen from an examinee. In FIG. 2A, the transmitting and receiving elements 1310 are arranged on the left side of an examinee in a prone position (on a side to which the left hand is stretched when seen from the examinee in the prone posture, i.e., the lower side of space). However, the transmitting and receiving elements 1310 may be arranged on the right side of an examinee in a prone position (on a side to which the right hand is stretched when seen from the examinee in the prone position, i.e., the upper side of the space). In the second acoustic wave receiving apparatus of FIGS. 2A and 2B, the transmitting and receiving elements 1310 are arranged on one of the left side and the right side of the array support unit 400. According to this configuration, the support member 1200 is not expanded to the tail side compared with a configuration including a simple hemispherical array support unit. Accordingly, the height of the support member 1200 from the lumber of an examinee to the tail side may be lowered. As a result, since an examinee makes a region from a hip joint to legs slightly lowered to support the entire body on the support member 1200, the examinee is allowed to take an easy posture.

(Third Arrangement)

In the third acoustic wave receiving apparatus of FIGS. 3A and 3B, the transmitting and receiving elements 1310 are arranged on both right and left sides of the array support unit 400. According to the configuration, an image of the left-half region of the subject portion E may be taken by the transmitting and receiving elements 1310 arranged on the left side, and an image of the right-half region of the subject portion E may be taken by the transmitting and receiving elements 1310 arranged on the right side. As a result, compared with the configuration in which the transmitting and receiving elements 1310 are arranged on one of the right and left sides as shown in FIGS. 2A and 2B, the moving distance of the scanner 500 in the right and left directions is remarkably reduced. In addition, compared with an acoustic wave receiving apparatus including a simple hemispherical array support unit, the moving distance of the scanner 500 in the right and left directions is only slightly increased.

According to the configuration of the third acoustic wave receiving apparatus, the expansion of the support member 1200 to the right and left sides does not impair the operability of an assisting person. Accordingly, the operability of an assisting person is not impaired. In addition, since the support member 1200 is not expanded to the tail side in the configuration of FIGS. 3A and 3B, the height of the support member 1200 from the lumber of an examinee to the tail side may be lowered. Therefore, the examinee is allowed to maintain an easy posture.

(Fourth Arrangement)

A configuration in which the transmitting and receiving elements 1310 are arranged on both the head side and the tail side of the array support unit 400 is also assumed. In this case, the upper half of a breast is measured by the transmitting and receiving elements on the head side, and the lower half of the breast is measured by the transmitting and receiving elements on the tail side. In this case, since an expansion amount of the support member 1200 to the tail side is suppressed, influence on the comfort of an examinee may be reduced. In addition, like the case of FIGS. 3A and 3B, a moving amount of the scanner 500 may be reduced. Moreover, since the support member 1200 is not expanded to the right and left sides in this configuration, the operability of an assisting person is favorably maintained. In the third arrangement or the fourth arrangement, an image of the half the subject portion E is taken by the transmitting and receiving elements 1310 on one side. Therefore, the third and fourth arrangements are suitable for alternately taking an image of the entire subject portion E with the receiving elements and the transmitting and receiving elements. Further, it may be possible to combine the third and fourth arrangements together to provide the transmitting and receiving elements at the right and left and the upper and lower (head and tail) four places of the subject portion E or provide the transmitting and receiving elements at angles other than right and left angles and upper and lower angles.

Furthermore, FIG. 1C describes a case in which the direction of the array support unit 400 itself with respect to the support member 1200 is fixed. However, the scanner 500 may have the function of changing a relative position in the rotating direction of the array support unit 400. By such a rotating and moving configuration, the spread of the moving region of the array support unit 400 may be restricted.

As described above, in an acoustic wave receiving apparatus capable of performing both photoacoustic imaging and ultrasound echo imaging, it is possible to enhance the comfort of the posture of an examinee while allowing an assisting person to favorably perform an assisting operation.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-094477, filed on May 10, 2016, and, Japanese Patent Application No. 2017-091603, filed on May 2, 2017, which are hereby incorporated by reference herein in their entirety.

What is claimed is:

1. An acoustic wave receiving apparatus comprising:
a light irradiation unit optically connected to a light source configured to irradiate a subject portion of an examinee with light;
a receiving array having a plurality of receiving elements configured to receive photoacoustic waves generated by an irradiation of the light from the light irradiation unit onto the subject portion and output photoacoustic signals;
a transmitting and receiving array having a plurality of transmitting and receiving elements configured to transmit ultrasound waves to the subject portion and receive echo waves reflected from the subject portion and then output ultrasound signals;
an array support unit having a first portion supporting the receiving array configured to direct a plurality of a reception directivity axes of the receiving elements toward an effective reception region and a second portion supporting the transmitting and receiving array;
a scanning unit configured to move the array support unit so as to scan the receiving array and the transmitting and receiving array integrally with respect to the subject portion; and
wherein the array support unit is so configured that the effective reception region and a focusing region formed by the transmitting and receiving array do not overlap each other.

2. The acoustic wave receiving apparatus according to claim 1, wherein
the second portion has a bottom surface part and a side surface part, and
the transmitting and receiving array is arranged at the bottom surface part.

3. The acoustic wave receiving apparatus according to claim 1, further comprising:
an acoustic-wave focusing unit that focuses the ultrasound waves, transmitted from the transmitting and receiving array, at a same depth as the effective reception region.

4. The acoustic wave receiving apparatus according to claim 1, wherein
the array support unit accommodates an acoustic matching fluid that acoustically couples the subject portion and the receiving array to each other and the subject portion and the transmitting and receiving array to each other.

5. The acoustic wave receiving apparatus according to claim 1, wherein
the scanning unit changes a relative position in a rotating direction of the array support unit with respect to the subject portion.

6. The acoustic wave receiving apparatus according to claim 1, wherein
the array support unit supports the transmitting and receiving array at a place closer to a head side than the receiving array.

7. The acoustic wave receiving apparatus according to claim 1, wherein
the array support unit supports the transmitting and receiving array on one of right and left sides of the receiving array.

8. The acoustic wave receiving apparatus according to claim 1, wherein
the array support unit supports the transmitting and receiving array on both right and left sides of the receiving array so as to allow ultrasound echo imaging for each half region of the subject portion.

9. The acoustic wave receiving apparatus according to claim 1, further comprising
a support member with an insertion opening configured to support the examinee and be inserted through with the subject portion of the examinee,
wherein a height of the support member on a tail side of the examinee is lower than a height of the support member on a head side thereof.

10. The acoustic wave receiving apparatus according to claim 1, further comprising:
a shape maintaining portion that maintains a shape of the subject portion.

11. The acoustic wave receiving apparatus according to claim 1, wherein
when an incident angle of the photoacoustic waves at which receiving sensitivity becomes 50% of a maximum value is α in each region of the receiving array, the transmitting and receiving array is arranged in a range in which an angle to be formed between the photoacoustic waves and receiving surfaces of the receiving elements is −α or less or α or more.

12. The acoustic wave receiving apparatus according to claim 1, wherein
the second portion of the array support unit is a region different from a region of the first portion.

13. The acoustic wave receiving apparatus according to claim 1, wherein
the second portion of the array support unit supports the transmitting and receiving array in a linear array shape.

14. The acoustic wave receiving apparatus according to claim 1, wherein
the effective reception region indicates an isocenter at where the plurality of the reception directivity axes of the receiving elements intersect commonly.

15. The acoustic wave receiving apparatus according to claim 1, further comprising;
an information processing unit that acquires characteristic information on the subject portion by using the photoacoustic signals and the ultrasound signals.

16. The acoustic wave receiving apparatus according to claim 1, wherein
the effective reception region to be formed by the plurality of receiving elements and a normal line of the receiving surface of the transmitting and receiving elements do not overlap each other.
